(19) United States
(12) Patent Application Publication
(10) Pub. No.: US 2002/0155511 A1
(43) Pub. Date: Oct. 24, 2002

(54) Novel antibody compositions for the negative selection of specific rat leukocyte subsets

(76) Inventors: Carolyn Horrocks, Stockholm (SE); Terry E. Thomas, Vancouver (CA)

Correspondence Address: John S. Pratt, Esq., KILPATRICK STOCKTON LLP, Suite 2800, 1100 Peachtree Street, Atlanta, GA, 30309-4530, US

(21) Appl. No.: 09/948,718

(22) Filed: Sep. 7, 2001

Related U.S. Application Data
(60) Provisional application No. 60/231,216, filed on Sep. 8, 2000.

(51) Int. Cl. G01N 33/567; G01N 33/53; G01N 33/555; C07K 16/28

(52) U.S. Cl. 435/7.21; 530/388.22

(57) ABSTRACT

Novel antibody compositions for the negative selection of rat cells are described.

This application claims the benefit under 35 USC §119(e) from U.S. Provisional patent application serial number 60/231,216, filed Sep. 8, 2000.

### FIELD OF THE INVENTION

The invention provides novel antibody compositions and negative selection processes for enriching subsets of rat cells from mixed hematopoietic cell suspensions.

### BACKGROUND OF THE INVENTION

The fields of hematology, immunology and cell biology rely on hematopoietic cell suspensions derived from samples of peripheral blood, spleen, bone marrow, lymph nodes, thymus and other related tissues. The separation of specific cell types from these heterogeneous samples is crucial to research in these fields. The rat is frequently used as a model of human physiology and disease and hence separation of rat cells is a requirement for many research projects.

Purified populations of immune cells, such as T cells and Antigen Presenting Cells (APC), are necessary for the study of immune mechanism. Primitive hematopoietic cells are isolated for the study and expansion of hematopoietic stem cells. Investigation of these cellular, molecular and biochemical processes require analyses of certain cell types in isolation. Numerous techniques have been used to isolate leukocyte subsets, including separation on the basis of physical characteristics such as density. The advent of monoclonal antibodies against cell surface antigens has greatly expanded the potential to distinguish and separate specific cell subsets. There are two basic approaches to cell separation using monoclonal antibodies. In positive selection techniques the desired cells are labeled with antibodies and removed from the remaining unwanted cells. In contrast, negative selection involves the labelling and removal of the unwanted cells from the suspension.

Treatment with antibody plus complement and the use of immunotoxins are both solely negative selection techniques. In contrast, FACS sorting and most batchwise immunoadsorption techniques can be adapted to both positive and negative selection. In immuno-adsorption techniques cells are selected with monoclonal antibodies and preferentially bound to a surface e.g. column of beads, flasks, magnetic particles. The bound, labelled cells can then be separated from the free unlabelled cells. Immuno-adsorption techniques are favoured as they combine the high specificity of targetting cells by monoclonal antibodies with the ability to be scaled up to accommodate large numbers of cells.

Binding of antibodies to molecules on the cell surface can have a profound influence on cell function. For example, binding of antibodies to CD3 on the T cell surface can be extremely mitogenic. Consequently, negative selection procedures are preferred as the desired cells are never labelled with antibody and hence their function is not perturbed. Negative selection therefore has a significant advantage over currently available immunomagnetic positive selection methods such as the system offered by Miltenyi Biotec (Bergisch Gladbach, Germany).

StemSep™ is an immunomagnetic negative selection system in which the unwanted cells are labelled with a cocktail of bispecific tetrameric antibody complexes (U.S. Pat. No. 5,514,340). The unwanted cells are then bound to magnetic colloid via the tetrameric antibody complexes and removed from the cell suspension in a high gradient magnetic separation column.

### SUMMARY OF THE INVENTION

The inventors have developed antibody compositions for use in enriching specific subsets of leukocytes (T cells, T cell subsets, B cells, NK cells), dendritic cells, monocytes and hematopoietic progenitors from rat hematopoietic cell suspensions including blood, bone marrow, spleen, thymus, liver and lymph nodes. The antibodies in the antibody composition are specific for selective markers associated with specific subsets of rat cells. The inventors have found that negative selection techniques using various antibody compositions consisting of antibodies specific for the unwanted rat hematopoietic cells gives a cell suspension highly enriched for the desired subset of rat cells. The cocktails in the invention specifically target each unwanted cell subsets which is a significant advantage over current immunoadsorption methods which rely on non-specific adherence to the column matrix for removal of the desired cells.

In one embodiment, the present invention provides a composition for enriching rat T cells comprising antibodies specific for the antigens (a) SIRP and/or CD11 b; (b) CD45RA and/or IgM and/or RNL9D3; and (c) CD161.

In another embodiment, the present invention provides a composition for enriching rat B cells comprising antibodies specific for the antigens (a) (i) TCRαβ, and/or (ii) CD5 and/or (iii) CD2 and/or (iv) CD3 and/or (v) CD28 and/or (vi) OX52 and/or (vii) CD4 and CD8; (b) SIRP and/or CD11b; and (c) CD161.

In a further embodiment, the present invention provides a composition for enriching rat NK cells comprising antibodies specific for the antigens (a) TCRαβ, and/or CD3 and/or CD28; (b) SIRP and/or CD11b; and (c) CD45RA and/or IgM and/or RNL9D3.

In yet another embodiment, the present invention provides a composition for enriching rat monocyte cells comprising antibodies specific for the antigens (a) TCRαβ and/or CD5 and/or CD2 and/or CD3 and/or OX52; (b) CD161; (c) CD45RA and/or IgM and/or RNL9D3; and (d) MOM/3F12/F2 and/or HIS48.

In yet a further embodiment, the present invention provides a composition for enriching rat dendritic cells comprising antibodies specific for the antigens (a) TCRαβ and/or CD5 and/or CD2 and/or CD3 and/or OX52; (b) CD161; (c) CD45RA and/or IgM and/or RNL9D3; and (d) ED1.

In a further embodiment, the present invention provides a composition for enriching rat hematopoietic progenitor cells comprising antibodies specific for the antigens (a) (i) TCRαβ and/or (ii) CD5 and/or (iii) CD2 and/or (iv) CD3 and/or (v) OX52 and/or (vi) CD4 and CD8; (b) CD161; (c) CD11b and/or SIRP; and (d) CD45RA and/or RNL9D3.

The present invention also includes negative selection processes for enriching and recovering rat cells from a rat hematopoietic cell suspension.

In one embodiment, the present invention relates to a negative selection process for enriching and recovering rat T cells in a sample comprising (1) reacting the sample with an antibody composition containing antibodies capable of binding to the antigens (a) SIRP and/or CD11b; (b) CD45RA and/or IgM and/or RNL9D3; and (c) CD161 under conditions permitting the formation of conjugates between the antibodies and cells in the sample having the antigens (a) SIRP and/or CD11b; (b) CD45RA and/or IgM and/or RNL9D3; and (c) CD161 on their surfaces; (2) removing the conjugates; and (3) recovering a cell preparation which is enriched in rat T cells.

In another embodiment, the present invention relates to a negative selection process for enriching and recovering rat B cells in a sample comprising (1) reacting the sample with an antibody composition containing antibodies capable of binding to the antigens (a) (i) TCRαβ and/or (ii) CD5 and/or (iii) CD2 and/or (iv) CD3 and/or (v) CD28 and/or (vi) OX52 and/or (vii) CD4 and CD8; (b) SIRP and/or CD11b; and (c) CD161 under conditions permitting the formation of conjugates between the antibodies and cells in the sample having the antigens (a) (i) TCRαβ and/or (ii) CD5 and/or (iii) CD2 and/or (iv) CD3 and/or (v) CD28 and/or (vi) OX52 and/or (vii) CD4 and CD8; (b) SIRP and/or CD11b; and (c) CD161 on their surfaces; (2) removing the conjugates; and (3) recovering a cell preparation which is enriched in rat B cells.

In a further embodiment, the present invention relates to a negative selection process for enriching and recovering rat NK cells in a sample comprising (1) reacting the sample with an antibody composition containing antibodies capable of binding to the antigens (a) TCRαβ and/or CD3 and/or CD28; (b) SIRP and/or CD11b; and (c) CD45RA and/or IgM and/or RNL9D3 under conditions permitting the formation of conjugates between the antibodies and cells in the sample having the antigens (a) TCRαβ and/or CD3 and/or CD28; (b) SIRP and/or CD11b; and (c) CD45RA and/or IgM and/or RNL9D3 on their surfaces; (2) removing the conjugates; and (3) recovering a cell preparation which is enriched in rat NK cells.

In yet another embodiment, the present invention relates to a negative selection process for enriching and recovering rat monocyte cells in a sample comprising (1) reacting the sample with an antibody composition containing antibodies capable of binding to the antigens (a) TCRαβ and/or CD5 and/or CD2 and/or CD3 and/or OX52; (b) CD161; (c) CD45RA and/or IgM and/or RNL9D3; and (d) MOM/3F12/F2 and/or HIS48 under conditions permitting the formation of conjugates between the antibodies and cells in the sample having the antigens (a) TCRαβ and/or CD5 and/or CD2 and/or CD3 and/or OX52; (b) CD161; (c) CD45RA and/or IgM and/or RNL9D3; and (d) MOM/3F12/F2 and/or HIS48 on their surfaces; (2) removing the conjugates; and (3) recovering a cell preparation which is enriched in rat monocyte cells.

In a further embodiment, the present invention relates to a negative selection process for enriching and recovering rat dendritic cells in a sample comprising (1) reacting the sample with an antibody composition containing antibodies capable of binding to the antigens (a) TCRαβ, and/or CD5 and/or CD2 and/or CD3 and/or OX52; (b) CD161; (c) CD45RA and/or IgM and/or RNL9D3; and (d) ED1 under conditions permitting the formation of conjugates between the antibodies and cells in the sample having the antigens (a) TCRαβ, and/or CD5 and/or CD2 and/or CD3 and or OX52; (b) CD161; (c) CD45RA and/or IgM and/or RNL9D3; and (d) ED1 on their surfaces; (2) removing the conjugates; and (3) recovering a cell preparation which is enriched in rat dendritic cells.

In yet another embodiment, the present invention relates to a negative selection process for enriching and recovering rat hematopoietic progenitor cells in a sample comprising (1) reacting the sample with an antibody composition containing antibodies capable of binding to the antigens (a) (i) TCRαβ and/or (ii) CD5 and/or (iii) CD2 and/or (iv) CD3 and/or (v) OX52 and/or (vi) CD4 and CD8; (b) CD161; (c) CD11b and/or SIRP; and (d) CD45RA and/or RNL9D3 under conditions permitting the formation of conjugates between the antibodies and cells in the sample having the antigens (a) (i) TCRαβ and/or (ii) CD5 and/or (iii) CD2 and/or (iv) CD3 and/or (v)OX52 and/or (vi) CD4 and CD8; (b) CD161; (c) CD11b and/or SIRP; and (d) CD45RA and/or RNL9D3 on their surfaces; (2) removing the conjugates; and (3) recovering a cell preparation which is enriched in rat hematopoietic progenitor cells.

The present invention also includes kits containing the antibody compositions of the invention together with instructions for the use thereof in preparing enriched rat cell populations.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a schematic drawing of the magnetic labelling of rat cells.

FIG. 2 is a FACS profile showing rat spleen cells prior to (A) and following (B) enrichment of CD3⁺ T cells using the method of the invention.

FIG. 3 is a FACS profile showing rat spleen cells prior to (A) and following (B) enrichment of CD4⁺ T cells using the method of the invention.

FIG. 4 is a FACS profile showing rat spleen cells prior to (A) and following (B) enrichment of CD8⁺ T cells using the method of the invention.

FIG. 5 is a FACS profile showing rat spleen cells prior to (A) and following (B) enrichment of B cells using the method of the invention.

FIG. 6 is a FACS profile showing rat spleen cells prior to (A) and following (B) enrichment of NK cells using the method of the invention.

### DETAILED DESCRIPTION OF THE INVENTION

I. Antibody Compositions

As hereinbefore mentioned, the present invention relates to antibody compositions for use in enriching specific subsets of rat leukocytes from rat cell suspensions.

In one embodiment, the present invention provides an antibody composition for enriching rat T cells comprising antibodies specific for the antigens (a) SIRP and/or CD11b; (b) CD45RA and/or IgM and/or RNL9D3; and (c) CD161. The T cell enrichment composition can optionally include antibodies to MOM/3F12/F2, OX62, HIS36, HIS48 and/or OX43. Preferably, the T cell enrichment composition comprises antibodies specific for SIRP, CD45RA, CD161 and OX43.

The present invention also include antibody compositions for the enrichment of T cell subsets such as CD4⁺ T cells, CD8⁺ T cells, naive CD4⁺ T cells, resting CD4⁺ T cells, resting CD8⁺ T cells, γδ T cells and αβ T cells.

Accordingly, in one embodiment the present invention provides a composition for enriching CD4⁺ T cells comprising antibodies specific for the antigens (a) CD8; (b) SIRP and/or CD11b; (c) CD45RA and/or IgM and/or RNL9D3; and (d) CD161. The CD4⁺ T cell enrichment composition can optionally include antibodies to MOM/3F12/F2, OX62, HIS36, HIS48, OX43. Preferably, the CD4⁺ T cell enrichment composition comprises antibodies specific for SIRP, CD45RA, CD161, OX43 and CD8.

In another embodiment, the present invention provides a composition for enriching CD8⁺ T cells and comprises antibodies specific for the antigens (a) CD4; (b) SIRP and/or CD11b; (c) CD45RA and/or IgM and/or RNL9D3; and (d) CD161. The CD8⁺ T cell enrichment composition can optionally include antibodies to MOM/3F12/F2, OX62, HIS36, HIS48 and/or OX43. Preferably, the CD8⁺ T cell enrichment composition comprises antibodies specific for SIRP, CD45RA, CD161, OX43 and CD4.

In a further embodiment, the present invention provides a composition for enriching naive CD4⁺ T cells and comprises antibodies specific for the antigens (a) CD8; (b) SIRP and/or CD11b; (c) CD45RA and/or IgM and/or RNL9D3; (d) CD161; and (e) CD45RC. The naive CD4⁺ T cell enrichment composition can optionally include antibodies to MOM/3F12/F2, OX62, HIS36, HIS48 and/or OX43.

In a further embodiment, the present invention provides a composition for enriching resting CD4⁺ T cells and comprises antibodies specific for the antigens (a) CD8; (b) SIRP and/or CD11b; (c) CD45RA and/or IgM and/or RNL9D3; (d) CD161; and (e) CD25 and/or CD45RC. The resting CD4⁺ T cell enrichment composition can optionally include antibodies to MOM/3F12/F2, OX62, HIS36, HIS48 and/or OX43.

In a further embodiment, the present invention provides a composition for enriching resting CD8⁺ T cells and comprises antibodies specific for the antigens (a) CD4; (b) SIRP and/or CD11b; (c) CD45RA and/or IgM and/or RNL9D3; (d) CD25; and (e) CD161. The resting CD8⁺ T cell enrichment composition can optionally include antibodies to MOM/3F12/F2, OX62, HIS36, HIS48 and/or OX43.

In a further embodiment, the present invention provides a composition for enriching γδ T cells and comprises antibodies specific for the antigens (a) TCRαβ; (b) SIRP and/or CD11b; (c) CD45RA and/or IgM and/or RNL9D3; and (d) CD161. The γδ T cell enrichment composition can optionally include antibodies to MOM/3F12/F2, OX62, HIS36, HIS48 and/or OX43.

In a further embodiment, the present invention provides a composition for enriching αβ T cells and comprises antibodies specific for the antigens (a) TCRγδ; (b) SIRP and/or CD11b; (c) CD45RA and/or IgM and/or RNL9D3; and (d) CD161. The αβ T cell enrichment composition can optionally include antibodies to MOM/3F12/F2, OX62, HIS36, HIS48 and/or OX43.

In another embodiment, the present invention provides a composition for enriching rat B cells comprising antibodies specific for the antigens (a) (i) TCRαβ, and/or (ii) CD5 and/or (iii) CD2 and/or (iv) CD3 and/or (v) CD28 and/or (vi) OX52 and/or (vii) CD4 and CD8; (b) SIRP and/or CD11b; and (c) CD161. The B cell enrichment composition can optionally include antibodies to MOM/3F12/F2, OX62, HIS36, HIS48 and/or OX43. Preferably, the B cell enrichment composition comprises antibodies specific for the antigens (a) (i) CD5 and/or (ii) CD4 and CD8 and/or (iii) CD28; (b) SIRP; (c) CD161 and (d) OX43.

In a further embodiment, the present invention provides a composition for enriching rat NK cells comprising antibodies specific for the antigens (a) TCRαβ and/or CD3 and/or CD28; (b) SIRP and/or CD11b; and (c) CD45RA and/or IgM and/or RNL9D3. The NK enrichment composition can optionally include antibodies to MOM/3F12/F2, OX62, HIS36, HIS48 and/or OX43. Preferably, the NK cell enrichment composition comprises antibodies specific for the antigens (a) CD5; (b) SIRP; (c) CD45RA and (d) OX43.

In yet another embodiment, the present invention provides a composition for enriching rat monocyte cells comprising antibodies specific for the antigens (a) TCRαβ and/or CD5 and/or CD2 and/or CD3 and/or OX52; (b) CD161; (c) CD45RA and/or IgM and/or RNL9D3; and (d) MOM/3F12/F2 and/or HIS48. The monocyte enrichment composition can optionally include antibodies to OX43 and/or HIS36.

In yet a further embodiment, the present invention provides a composition for enriching rat dendritic cells comprising antibodies specific for the antigens (a) TCRαβ and/or CD5 and/or CD2 and/or CD3 and or OX52; (b) CD161; (c) CD45RA and/or IgM and/or RNL9D3; and (d) ED1. The dendritic cell enrichment composition can optionally include antibodies to OX43, HIS48 and/or MOM/3F12/F2.

In a further embodiment, the present invention provides a composition for enriching rat hematopoietic progenitor cells comprising antibodies specific for the antigens (a) (i) TCRαβ and/or (ii) CD5 and/or (iii) CD2 and/or (iv) CD3 and/or (v) OX52 and/or (vi) CD4 and CD8; (b) CD161; (c) CD11b and/or SIRP; and (d) CD45RA and/or RNL9D3. The progenitor cell enrichment composition can optionally include OX62, OX43, MOM/3F12/F2, HIS36 and/or HIS48.

One skilled in the art will appreciate that in addition to the antibodies listed above, the antibody compositions may additionally include other antibodies that are specific for antigens on the surface of the rat cells you wish to deplete from the sample including those listed in Tables 1 and 2. The selection of the antibodies can depend on many factors including the nature of the sample to be enriched.

Within the context of the present invention, antibodies are understood to include monoclonal antibodies and polyclonal antibodies, antibody fragments (e.g., Fab, and F(ab′)₂) and chimeric antibodies. Antibodies are understood to be reactive against a selected antigen on the surface of a cell if they bind with an appropriate affinity (association constant), e.g. greater than or equal to 10⁷ M^{-1.}

Polyclonal antibodies against selected antigens on the surface of unwanted cells may be readily generated by one of ordinary skill in the art from a variety of warm-blooded animals such as horses, cows, various fowl, rabbits, mice, hamsters, or rats. For example, a mammal, (e.g., a mouse, hamster, or rabbit) can be immunized with an immunogenic form of an antigen which elicits an antibody response in the mammal. Techniques for conferring immunogenicity on an antigen include conjugation to carriers or other techniques well known in the art. For example, the antigen can be administered in the presence of adjuvant. The progress of immunization can be monitored by detection of antibody titers in plasma or serum. Following immunization, antisera can be obtained and polyclonal antibodies isolated from the sera.

Monoclonal antibodies are preferably used in the antibody compositions of the invention. Monoclonal antibodies specific for selected antigens on the surface of unwanted cells may be readily generated using conventional techniques. For example, monoclonal antibodies may be produced by the hybridoma technique originally developed by Kohler and Milstein 1975 (Nature 256, 495-497; see also U.S. Pat. Nos. RE 32,011, 4,902,614, 4,543,439, and 4,411,993 which are incorporated herein by reference; see also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988). Other techniques may also be utilized to construct monoclonal antibodies (for example, see William D. Huse et al., 1989, “Generation of a Large Combinational Library of the Immunoglobulin Repertoire in Phage Lambda,” Science 246:1275-1281, L. Sastryet al., 1989 “Cloning of the Immunological Repertoire in Escherichia coli for Generation of Monoclonal Catalytic Antibodies: Construction of a Heavy Chain Variable Region-Specific cDNA Library,” Proc Natl. Acad. Sci USA 86:5728-5732; Kozbor et al., 1983 Immunol. Today 4, 72 re the human B-cell hybridoma technique; Cole et al. 1985 Monoclonal Antibodies in Cancer Therapy, Allen R. Bliss, Inc., pages 77-96 re the EBV-hybridoma technique to produce human monoclonal antibodies; and see also Michelle Alting-Mees et al., 1990 “Monoclonal Antibody Expression Libraries: A Rapid Alternative to Hybridomas,” Strategies in Molecular Biology 3:1-9). Hybridoma cells can be screened immunochemically for production of antibodies specifically reactive with an antigen, and monoclonal antibodies can be isolated.

The term “antibody” as used herein is intended to include antibody fragments which are specifically reactive with specific antigens on the surface of unwanted cells. Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments.

The invention also contemplates chimeric antibody derivatives, i.e., antibody molecules that combine a non-human animal variable region and a human constant region. Chimeric antibody molecules can include, for example, the antigen binding domain from an antibody of a mouse, rat, or other species, with human constant regions. A variety of approaches for making chimeric antibodies have been described and can be used to make chimeric antibodies containing the immunoglobulin variable region which recognizes selected antigens on the surface of differentiated cells or tumor cells. See, for example, Morrison et al., 1985; Proc. Natl. Acad. Sci. U.S.A. 81,6851; Takeda et al., 1985, Nature 314:452; Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B.

Antibodies may be selected for use in the antibody compositions of the invention based on their ability to deplete targeted unwanted cells and recover non-targeted cells in magnetic cell separations as more particularly described herein, and in U.S. Pat. No. 5,514,340, which is incorporated in its entirety herein by reference.

II. Processes For Preparing Enriched Rat Cell Preparations

The antibody compositions of the invention may be used to isolate suspensions enriched for particular rat cell subsets. The present invention also includes negative selection processes for enriching and recovering rat cells from a rat hematopoietic cell suspension. In accordance with a process of the invention, a sample is reacted with an antibody composition containing antibodies which are specific for selected antigens on the surface of the unwanted cells to be removed from the sample and not on the cells to be enriched in the sample, under suitable conditions, conjugates form between the antibodies contained in the antibody composition and the cells in the sample containing the antigens on their surface; and the conjugates are removed to provide a cell preparation enriched in specific cells. The antibody compositions for use in the negative selection method may be as described above in Section (I) or in Table 2.

In one embodiment, the present invention relates to a negative selection process for enriching and recovering rat T cells in a sample comprising (1) reacting the sample with an antibody composition containing antibodies capable of binding to the antigens (a) SIRP and/or CD11b; (b) CD45RA and/or IgM and/or RNL9D3; and (c) CD161 under conditions permitting the formation of conjugates between the antibodies and cells in the sample having the antigens (a) SIRP and/or CD11b; (b) CD45RA and/or IgM and/or RNL9D3; and (c) CD161 on their surfaces; (2) removing the conjugates; and (3) recovering a cell preparation which is enriched in rat T cells.

In another embodiment, the present invention relates to a negative selection process for enriching and recovering rat CD4⁺ T cells in a sample comprising (1) reacting the sample with an antibody composition containing antibodies capable of binding to the antigens (a) CD8; (b) SIRP and/or CD11b; (c) CD45RA and/or IgM and/or RNL9D3; and (d) CD161 under conditions permitting the formation of conjugates between the antibodies and cells in the sample having the antigens (a) CD8; (b) SIRP and/or CD11b; (c) CD45RA and/or IgM and/or RNL9D3; and (d) CD161 on their surfaces; (2) removing the conjugates; and (3) recovering a cell preparation which is enriched in rat CD4⁺ T cells.

In another embodiment, the present invention relates to a negative selection process for enriching and recovering rat CD8⁺ T cells in a sample comprising (1) reacting the sample with an antibody composition containing antibodies capable of binding to the antigens (a) CD4; (b) SIRP and/or CD11b; (c) CD45RA and/or IgM and/or RNL9D3; and (d) CD161 under conditions permitting the formation of conjugates between the antibodies and cells in the sample having the antigens (a) CD4; (b) SIRP and/or CD11b; (c) CD45RA and/or IgM and/or RNL9D3; and (d) CD161 on their surfaces; (2) removing the conjugates; and (3) recovering a cell preparation which is enriched in rat CD8⁺ T cells.

In another embodiment, the present invention relates to a negative selection process for enriching and recovering rat naive CD4⁺ T cells in a sample comprising (1) reacting the sample with an antibody composition containing antibodies capable of binding to the antigens (a) CD8; (b) SIRP and/or CD11b; (c) CD45RA and/or IgM and/or RNL9D3; (d) CD161; and (e) CD45RC under conditions permitting the formation of conjugates between the antibodies and cells in the sample having the antigens (a) CD8; (b) SIRP and/or CD11b; (c) CD45RA and/or IgM and/or RNL9D3; (d) CD161; and (e) CD45RC on their surfaces; (2) removing the conjugates; and (3) recovering a cell preparation which is enriched in rat naive CD4⁺ T cells.

In another embodiment, the present invention relates to a negative selection process for enriching and recovering rat resting CD4⁺ T cells in a sample comprising (1) reacting the sample with an antibody composition containing antibodies capable of binding to the antigens (a) CD8; (b) SIRP and/or CD11b; (c) CD45RA and/or IgM and/or RNL9D3; (d) CD161; and (e) CD25 and/or CD45RC under conditions permitting the formation of conjugates between the antibodies and cells in the sample having the antigens (a) CD8; (b) SIRP and/or CD11b; (c) CD45RA and/or IgM and/or RNL9D3; (d) CD161; and (e) CD25 and/or CD45RC on their surfaces; (2) removing the conjugates; and (3) recovering a cell preparation which is enriched in rat resting CD4⁺ T cells.

In another embodiment, the present invention relates to a negative selection process for enriching and recovering rat resting CD8⁺ T cells in a sample comprising (1) reacting the sample with an antibody composition containing antibodies capable of binding to the antigens (a) CD4; (b) SIRP and/or CD11b; (c) CD45RA and/or IgM and/or RNL9D3; (d) CD25; and (e) CD161 under conditions permitting the formation of conjugates between the antibodies and cells in the sample having the antigens (a) CD4; (b) SIRP and/or CD11b; (c) CD45RA and/or IgM and/or RNL9D3; (d) CD25; and (e) CD161 on their surfaces; (2) removing the conjugates; and (3) recovering a cell preparation which is enriched in rat resting CD8⁺ T cells.

In another embodiment, the present invention relates to a negative selection process for enriching and recovering rat γδ T cells in a sample comprising (1) reacting the sample with an antibody composition containing antibodies capable of binding to the antigens (a) TCRαβ; (b) SIRP and/or CD11b; (c) CD45RA and/or IgM and/or RNL9D3; and (d) CD161 under conditions permitting the formation of conjugates between the antibodies and cells in the sample having the antigens (a) TCRαβ; (b) SIRP and/or CD11b; (c) CD45RA and/or IgM and/or RNL9D3; and (d) CD161 on their surfaces; (2) removing the conjugates; and (3) recovering a cell preparation which is enriched in rat γδ T cells.

In another embodiment, the present invention relates to a negative selection process for enriching and recovering rat αβ T cells in a sample comprising (1) reacting the sample with an antibody composition containing antibodies capable of binding to the antigens (a) TCRγδ; (b) SIRP and/or CD11b; (c) CD45RA and/or IgM and/or RNL9D3; and (d) CD161 under conditions permitting the formation of conjugates between the antibodies and cells in the sample having the antigens (a) TCRγδ; (b) SIRP and/or CD11b; (c) CD45RA and/or IgM and/or RNL9D3; and (d) CD161 on their surfaces; (2) removing the conjugates; and (3) recovering a cell preparation which is enriched in rat αβ T cells.

In another embodiment, the present invention relates to a negative selection process for enriching and recovering rat B cells in a sample comprising (1) reacting the sample with an antibody composition containing antibodies capable of binding to the antigens (a) (i) TCRαβ and/or (ii) CD5 and/or (iii) CD2 and/or (iv) CD3 and/or (v) CD28 and/or (vi) OX52 and/or (vii) CD4 and CD8; (b) SIRP and/or CD11b; and (c) CD161 under conditions permitting the formation of conjugates between the antibodies and cells in the sample having the antigens (a) (i) TCRαβ and/or (ii) CD5 and/or (iii) CD2 and/or (iv) CD3 and/or (v) CD28 and/or (vi) OX52 and/or (vii) CD4 and CD8; (b) SIRP and/or CD11b; and (c) CD161 on their surfaces; (2) removing the conjugates; and (3) recovering a cell preparation which is enriched in rat B cells.

In a further embodiment, the present invention relates to a negative selection process for enriching and recovering rat NK cells in a sample comprising (1) reacting the sample with an antibody composition containing antibodies capable of binding to the antigens (a) TCRαβ and/or CD3 and/or CD28; (b) SIRP and/or CD11b; and (c) CD45RA and/or IgM and/or RNL9D3 under conditions permitting the formation of conjugates between the antibodies and cells in the sample having the antigens (a) TCRαβ and/or CD3 and/or CD28; (b) SIRP and/or CD11b; and (c) CD45RA and/or IgM and/or RNL9D3 on their surfaces; (2) removing the conjugates; and (3)-recovering a cell preparation which is enriched in rat NK cells.

In yet another embodiment, the present invention relates to a negative selection process for enriching and recovering rat monocyte cells in a sample comprising (1) reacting the sample with an antibody composition containing antibodies capable of binding to the antigens (a) TCRαβ and/or CD5 and/or CD2 and/or CD3 and/or OX52; (b) CD161; (c) CD45RA and/or IgM and/or RNL9D3; and (d) MOM/3F12/F2 and/or HIS48 under conditions permitting the formation of conjugates between the antibodies and cells in the sample having the antigens (a) TCRαβ and/or CD5 and/or CD2 and/or CD3 and/or OX52; (b) CD161; (c) CD45RA and/or IgM and/or RNL9D3; and (d) MOM/3F12/F2 and/or HIS48 on their surfaces; (2) removing the conjugates; and (3) recovering a cell preparation which is enriched in rat monocyte cells.

In a further embodiment, the present invention relates to a negative selection process for enriching and recovering rat dendritic cells in a sample comprising (1) reacting the sample with an antibody composition containing antibodies capable of binding to the antigens (a) TCRαβ and/or CD5 and/or CD2 and/or CD3 and/or OX52; (b) CD161; (c) CD45RA and/or IgM and/or RNL9D3; and (d) ED1 under conditions permitting the formation of conjugates between the antibodies and cells in the sample having the antigens (a) TCRαβ and/or CD5 and/or CD2 and/or CD3 and or OX52; (b) CD161; (c) CD45RA and/or IgM and/or RNL9D3; and (d) ED1 on their surfaces; (2) removing the conjugates; and (3) recovering a cell preparation which is enriched in rat dendritic cells.

In yet another embodiment, the present invention relates to a negative selection process for enriching and recovering rat hematopoietic progenitor cells in a sample comprising (1) reacting the sample with an antibody composition containing antibodies capable of binding to the antigens (a) (i) TCRαβ and/or (ii) CD5 and/or (iii) CD2 and/or (iv) CD3 and/or (v) OX52 and/or (vi) CD4 and CD8; (b) CD161; (c) CD11b and/or SIRP; and (d) CD45RA and/or RNL9D3 under conditions permitting the formation of conjugates between the antibodies and cells in the sample having the antigens (a) (i) TCRαβ and/or (ii) CD5 and/or (iii) CD2 and/or (iv) CD3 and/or (v) OX52 and/or (vi) CD4 and CD8; (b) CD161; (c) SIRP and/or CD11b; and (d) CD45RA and/or RNL9D3 on their surfaces; (2) removing the conjugates; and (3) recovering a cell preparation which is enriched in rat hematopoietic progenitor cells.

In the above negative selection processes of the invention for rat cell enrichment, conditions which permit the formation of conjugates may be selected having regard to factors such as the nature and amounts of the antibodies in the antibody composition, and the estimated concentration of targeted cells in the sample.

The antibodies in the antibody compositions may be labelled with a marker or they may be conjugated to a matrix. Examples of markers are biotin, which can be removed by avidin bound to a support, and fluorochromes, e.g. fluorescein, which provide for separation using fluorescence activated sorters. Examples of matrices are magnetic beads, which allow for direct magnetic separation (Kemshead 1992), panning surfaces e.g. plates, (Lebkowski, J. S, et al., (1994), J. of Cellular Biochemistry supple. 18b:58), dense particles for density centrifugation (Van Vlasselaer, P., Density Adjusted Cell Sorting (DACS), A Novel Method to Remove Tumor Cells From Peripheral Blood and Bone Marrow StemCell Transplants. (1995) 3rd International Symposium on Recent Advances in Hematopoietic Stem Cell Transplantation-Clinical Progress, New Technologies and Gene Therapy, San Diego, Calif.), dense particles alone (Zwerner et al., Immunol. Meth. 1996 198(2):199-202) adsorption columns (Berenson et al. 1986, Journal of Immunological Methods 91:11-19.), and adsorption membranes. The antibodies may also be joined to a cytotoxic agent such as complement or a cytotoxin, to lyse or kill the targeted non-T cells.

The antibodies in the antibody compositions may be directly or indirectly coupled to a matrix. For example, the antibodies in the compositions of the invention may be chemically bound to the surface of magnetic particles for example, using cyanogen bromide. When the magnetic particles are reacted with a sample, conjugates will form between the magnetic particles with bound antibodies (specific for antigens on the surfaces of the cells) and the cells having the antigens on their surfaces.

Alternatively, the antibodies may be indirectly conjugated to a matrix using antibodies. For example, a matrix may be coated with a second antibody having specificity for the antibodies in the antibody composition. By way of example, if the antibodies in the antibody composition are mouse IgG antibodies, the second antibody may be rabbit anti-mouse IgG.

The antibodies in the antibody compositions may also be incorporated in antibody reagents which indirectly conjugate to a matrix. Examples of antibody reagents are bispecific antibodies, tetrameric antibody complexes, and biotinylated antibodies.

Bispecific antibodies contain a variable region of an antibody in an antibody composition of the invention, and a variable region specific for at least one antigen on the surface of a matrix. The bispecific antibodies may be prepared by forming hybrid hybridomas. The hybrid hybridomas may be prepared using the procedures known in the art such as those disclosed in Staerz & Bevan, (1986, PNAS (USA) 83: 1453) and Staerz & Bevan, (1986, Immunology Today, 7:241). Bispecific antibodies may also be constructed by chemical means using procedures such as those described by Staerz et al., (1985, Nature, 314:628) and Perez et al., (1985 Nature 316:354), or by expression of recombinant immunoglobulin gene constructs.

A tetrameric immunological complex may be prepared by mixing a first monoclonal antibody which is capable of binding to at least one antigen on the surface of a matrix, and a second monoclonal antibody from the antibody composition of the invention. The first and second monoclonal antibody are from a first animal species. The first and second antibody are reacted with an about equimolar amount of monoclonal antibodies of a second animal species which are directed against the Fc-fragments of the antibodies of the first animal species. The first and second antibody may also be reacted with an about equimolar amount of the F(ab′)₂ fragments of monoclonal antibodies of a second animal species which are directed against the Fc-fragments of the antibodies of the first animal species. (See U.S. Pat. No. 4,868,109 to Lansdorp, which is incorporated herein by reference for a description of tetrameric antibody complexes and methods for preparing same).

The antibodies of the invention may be biotinylated and indirectly conjugated to a matrix which is labelled with (strept) avidin. For example, biotinylated antibodies contained in the antibody composition of the invention may be used in combination with magnetic iron-dextran particles that are covalently labelled with (strept) avidin (Miltenyi, S. et al., Cytometry 11:231, 1990). Many alternative indirect ways to specifically cross-link the antibodies in the antibody composition and matrices would also be apparent to those skilled in the art.

In an embodiment of the invention, the cell conjugates are removed by magnetic separation using magnetic particles. Suitable magnetic particles include particles in ferrofluids and other colloidal magnetic solutions. “Ferrofluid” refers to a colloidal solution containing particles consisting of a magnetic core, such as magnetite (Fe₃O₄) coated or embedded in material that prevents the crystals from interacting. Examples of such materials include proteins, such as ferritin, polysaccharides, such as dextrans, or synthetic polymers such as sulfonated polystyrene cross-linked with divinylbenzene. The core portion is generally too small to hold a permanent magnetic field. The ferrofluids become magnetized when placed in a magnetic field. Examples of ferrofluids and methods for preparing them are described by Kemshead J. T. (1992) in J. Hematotherapy, 1:35-44, at pages 36 to 39, and Ziolo et al. Science (1994) 257:219 which are incorporated herein by reference. Colloidal particles of dextran-iron complex are preferably used in the process of the invention. (See Molday, R. S. and McKenzie, L. L. FEBS Lett. 170:232, 1984; Miltenyi et al., Cytometry 11:231, 1990; and Molday, R. S. and MacKenzie, D., J. Immunol. Methods 52:353, 1982; Thomas et al., J. Hematother. 2:297 (1993); and U.S. Pat. No. 4,452,733, which are each incorporated herein by reference).

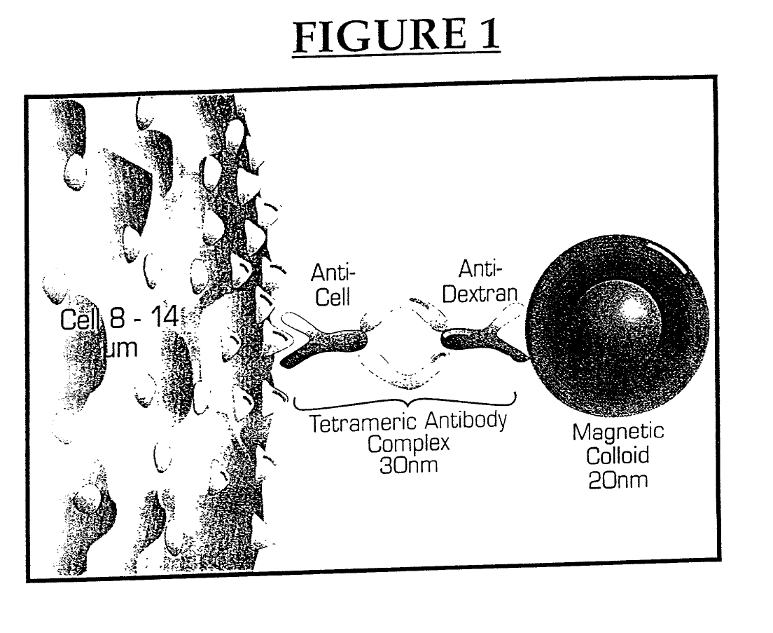

FIG. 1 is a schematic representation of magnetic cell labeling using tetrameric antibody complexes and colloidal dextran iron.

In accordance with the magnetic separation method, the sample containing the rat cells to be recovered, is reacted with the above described antibody reagents, preferably tetrameric antibody complexes, so that the antibody reagents bind to the unwanted cells present in the sample to form cell conjugates of the targeted unwanted cells and the antibody reagents. The reaction conditions are selected to provide the desired level of binding of the targeted cells and the antibody reagents. Preferably the sample is incubated with the antibody reagents for a period of 5 to 60 minutes at either 4° or ambient room temperature. The concentration of the antibody reagents is selected depending on the estimated concentration of the targeted differentiated cells in the sample. Generally, the concentration is between about 0.1 to 50 μg/ml of sample. The magnetic particles are then added and the mixture is incubated for a period of about 5 minutes to 30 minutes at the selected temperature. The sample is then ready to be separated over a magnetic filter device. Preferably, the magnetic separation procedure is carried out using the magnetic filter and methods described in U.S. Pat. No. 5,514,340 to Lansdorp and Thomas which is incorporated in its entirety herein by reference.

The sample containing the magnetically labelled cell conjugates is passed through the magnetic filter in the presence of a magnetic field. In a preferred embodiment of the invention, the magnet is a dipole magnet with a gap varying from 0.3 to 3.0 inches bore and having a magnetic field of 0.5-2 Tesla. The magnetically labelled cell conjugates are retained in the high gradient magnetic column and the materials which are not magnetically labelled flow through the column after washing with a buffer.

The preparation containing non-magnetically labelled cells may be analyzed using procedures such as flow cytometry.

IIII. Uses of the Composition of the Invention

The composition and process of the invention may be used in the processing of samples including spleen, thymus, blood, bone marrow, and lymph nodes from rats. Using the invention it is possible to recover highly purified rat cell subsets for the purpose of studying immune function development and pathophysiology, responses to infection, transplant biology, autoimmunity, the biology of hematopoietic stem cells and rat models of human diseases.

IV. Kits

The present invention also includes kits containing the antibody compositions of the invention together with instructions for the use thereof in preparing enriched rat cell populations.

The present invention also relates to a kit useful in preparing a cell preparation enriched in rat T cells comprising antibodies specific for the antigens (a) SIRP and/or CD11b; (b) CD45RA and/or IgM and/or RNL9D3; and (c) CD161, and instructions for preparing a cell preparation enriched in rat T cells. Preferably, the kit comprises antibodies specific for SIRP, CD45RA, CD161 and OX43.

The present invention also relates to a kit useful in preparing a cell preparation enriched in CD4⁺ T cells comprising antibodies specific for the antigens (a) CD8; (b) SIRP and/or CD11b; (c) CD45RA and/or IgM and/or RNL9D3; and (d) CD161, and instructions for preparing a cell preparation enriched in CD4⁺ T cells.

The present invention also relates to a kit useful in preparing a cell preparation enriched in CD8⁺ T cells comprising antibodies specific for the antigens (a) CD4; (b) SIRP and/or CD11b; (c) CD45RA and/or IgM and/or RNL9D3; and (d) CD161, and instructions for preparing a cell preparation enriched in CD8⁺ T cells.

The present invention also relates to a kit useful in preparing a cell preparation enriched in naive CD4⁺ T cells comprising antibodies specific for the antigens (a) CD8; (b) SIRP and/or CD11b; (c) CD45RA and/or IgM and/or RNL9D3; (d) CD161; and (e) CD45RC, and instructions for preparing a cell preparation enriched in naive CD4⁺ T cells.

The present invention also relates to a kit useful in preparing a cell preparation enriched in resting CD4⁺ T cells comprising antibodies specific for the antigens (a) CD8; (b) SIRP and/or CD11b; (c) CD45RA and/or IgM and/or RNL9D3; (d) CD161; and (e) CD25 and/or CD45RC, and instructions for preparing a cell preparation enriched in resting CD4⁺ T cells.

The present invention also relates to a kit useful in preparing a cell preparation enriched in resting CD8⁺ T cells comprising antibodies specific for the antigens (a) CD4; (b) SIRP and/or CD11b; (c) CD45RA and/or IgM and/or RNL9D3; (d) CD25; and (e) CD161, and instructions for preparing a cell preparation enriched in resting CD8⁺ T cells.

The present invention also relates to a kit useful in preparing a cell preparation enriched in γδ T cells comprising antibodies specific for the antigens (a) TCRαβ; (b) SIRP and/or CD11b; (c) CD45RA and/or IgM and/or RNL9D3; and (d) CD161, and instructions for preparing a cell preparation enriched in γδ T cells.

The present invention also relates to a kit useful in preparing a cell preparation enriched in αβ T cells comprising antibodies specific for the antigens (a) TCRγδ; (b) SIRP and/or CD11b; (c) CD45RA and/or IgM and/or RNL9D3; and (d) CD161, and instructions for preparing a cell preparation enriched in αβ T cells.

The present invention further relates to a kit useful in preparing a cell preparation enriched in rat B cells comprising antibodies specific for the antigens (a) (i) TCRαβ and/or (ii) CD5 and/or (iii) CD2 and/or (iii) CD3 and/or (v) CD28 and/or (vi) OX52 and/or (vii) CD4 and CD8; (b) SIRP and/or CD11b; and (c) CD161, and instructions for preparing a cell preparation enriched in rat B cells. Preferably, the kit comprises antibodies specific for the antigens (a) (i) CD5 and/or (ii) CD4 and CD8 and/or (iii) CD28; (b) SIRP; (c) CD161 and (d) OX43.

The present invention also relates to a kit useful in preparing a cell preparation enriched in rat NK cells comprising antibodies specific for the antigens (a) TCRαβ and/or CD3 and/or CD28; (b) SIRP and/or CD11b; and (c) CD45RA and/or IgM and/or RNL9D3, and instructions for preparing a cell preparation enriched in rat NK cells. Preferably, the kit comprises antibodies specific for the antigens (a) CD5; (b) SIRP; (c) CD45RA and (d) OX43.

The present invention yet also relates to a kit useful in preparing a cell preparation enriched in rat monocyte cells comprising antibodies specific for the antigens (a) TCRαβ and/or CD5 and/or CD2 and/or CD3 and/or OX52; (b) CD161; (c) CD45RA and/or IgM and/or RNL9D3; and (d) MOM/3F12/F2 and/or HIS48, and instructions for preparing a cell preparation enriched in rat monocyte cells.

The present invention further relates to a kit useful in preparing a cell preparation enriched in rat dendritic cells comprising antibodies specific for the antigens (a) TCRαβ and/or CD5 and/or CD2 and/or CD3 and or OX52; (b) CD161; (c) CD45RA and/or IgM and/or RNL9D3; and (d) ED1, and instructions for preparing a cell preparation enriched in rat dendritic cells.

The present invention also relates to a kit useful in preparing a cell preparation enriched in rat hematopoietic progenitor cells comprising antibodies specific for the antigens (a) (i) TCRαβ and/or (ii) CD5 and/or (iii) CD2 and/or (iv) CD3 and/or (v) OX52 and/or (vi) CD4 and CD8; (b) CD161; (c) CD11b and/or SIRP; and (d) CD45RA and/or RNL9D3, and instructions for preparing a cell preparation enriched in rat hematopoietic progenitor cells.

The following non-limiting examples are illustrative of the present invention:

### EXAMPLES

### Example 1

Method of Evaluating Antibody Combinations

Suspensions of rat spleen, blood and bone marrow cells were labelled with tetrameric antibodies and colloidal dextran iron for magnetic cell depletions. Murine IgG₁ monoclonal antibodies recognizing rat lineage specific cell surface antigens were mixed with a mouse IgG₁ anti-dextran antibody (Thomas, T. E, et al. (1992), J. Immunol Methods 154:245;252) and a rat IgG₁ monoclonal antibody which recognizes the Fc portion of the mouse IgG₁ molecule (TFL-P9) (Lansdorp, P. M, and Thomas, T. E. (1990), Mol. Immunol. 27:659-666). Tetrameric antibody complexes (Lansdorp, P. M, and Thomas, T. E. (1990), Mol. Immunol. 27:659-666; U.S. Pat. No. 4,868,109 to Lansdorp) spontaneously form when mouse IgG₁ molecules (the lineage specific monoclonal antibody and anti-dextran monoclonal antibody) are mixed with TFL-P9. A proportion of these tetrameric antibody complexes are bifunctional, recognizing an antigen on the surface of the target cell on one side and dextran (part of the magnetic colloidal anti-dextran monoclonal antibody) on the other. Tetrameric antibody complexes were made for all the antibodies in the lineage cocktail. FIG. 1 shows a schematic representation of magnetic cell labeling using tetrameric antibody complexes and colloidal dextran iron.

Cells were labelled for separation (1-5×10⁷ cells/ml) by incubating them with the desired combination of tetramers for 30 min on ice followed by a 30 min incubation with colloidal dextran iron (final OD450=0.6) (Molday and MacKenzie 1982, 52(3): 353-367). The cells were then passed through a magnetic filter (U.S. Pat. No. 5,514,340; inventors Lansdorp and Thomas) at 1 cm/min. The magnetically labelled cells bind to the filter and the unlabeled cells pass through. FIG. 1 shows a schematic representation of magnetic cell labeling using tetrameric antibody complexes and colloidal dextran iron.

The flow through fraction is collected and analyzed for the frequency of desired cells using Flow Cytometry. The enrichment of each cell type depends on how well the antibody cocktail has targeted other cells for removal. Each antibody cocktail was evaluated for the purity and recovery of the desired cell type.

### Example 2

Isolation of CD3⁺ T Cell from Rat Spleen

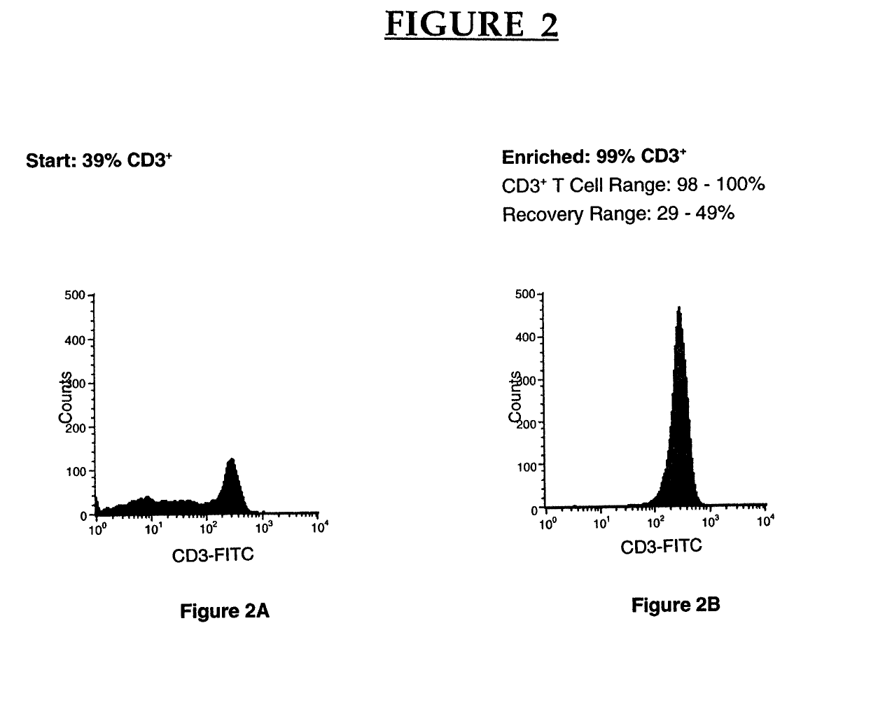

This example demonstrates the enrichment of T cells from splenocytes using the method described in Example 1. AT cell enrichment cocktail containing antibodies against OX43, CD45R, CD161 and SIRP was prepared. The results shown in FIG. 2 demonstrate that the method of the invention results in 99% purity of CD3⁺ T cells with a recovery of 35%.

### Example 3

Isolation of CD4⁺ T Cell from Rat Spleen

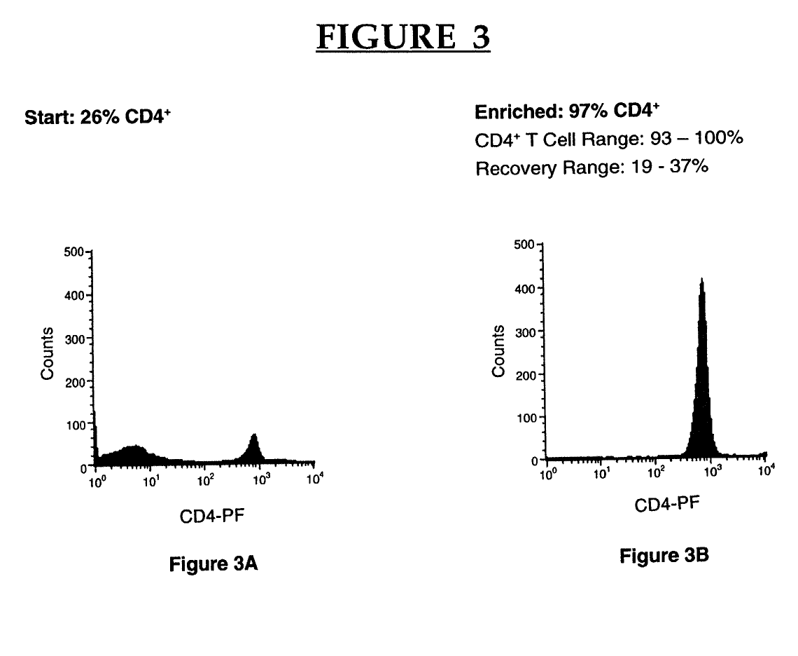

This example demonstrates the enrichment of T cells from splenocytes using the method described in Example 1. A T cell enrichment cocktail containing antibodies against OX43, CD45R, CD161, SIRP and CD8 was prepared. The results shown in FIG. 3 demonstrate that the method of the invention results in 95% purity of CD4⁺ T cells with a recovery of 30%.

### Example 4

Isolation of CD8⁺ T Cell from Rat Spleen

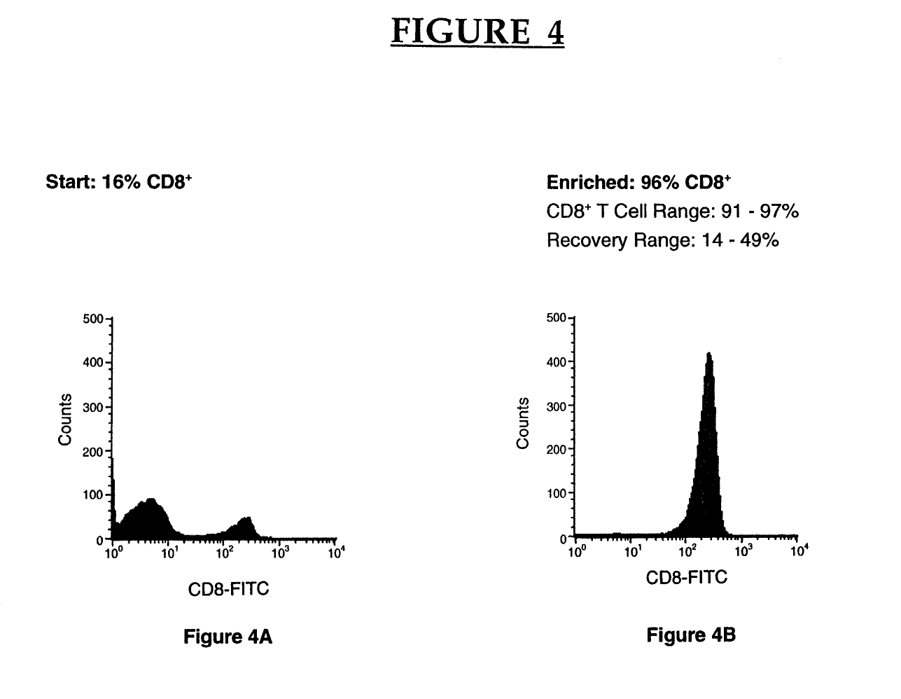

This example demonstrates the enrichment of T cells from splenocytes using the method described in Example 1. A T cell enrichment cocktail containing antibodies against OX43, CD45R, CD161, CD4 and SIRP was prepared. The results shown in FIG. 4 demonstrate that the method of the invention results in 95% purity of CD8⁺ T cells with a recovery of 30%.

### Example 5

Reproducibility of Rat T Cell Isolations

This example demonstrates the reproducibility (n=8) of Rat T cell separations from splenocytes using the method described in Example 1. Results of eight replicate separations are shown in Table 3.

### Example 6

Isolation of B cells (panB) from Rat Spleen

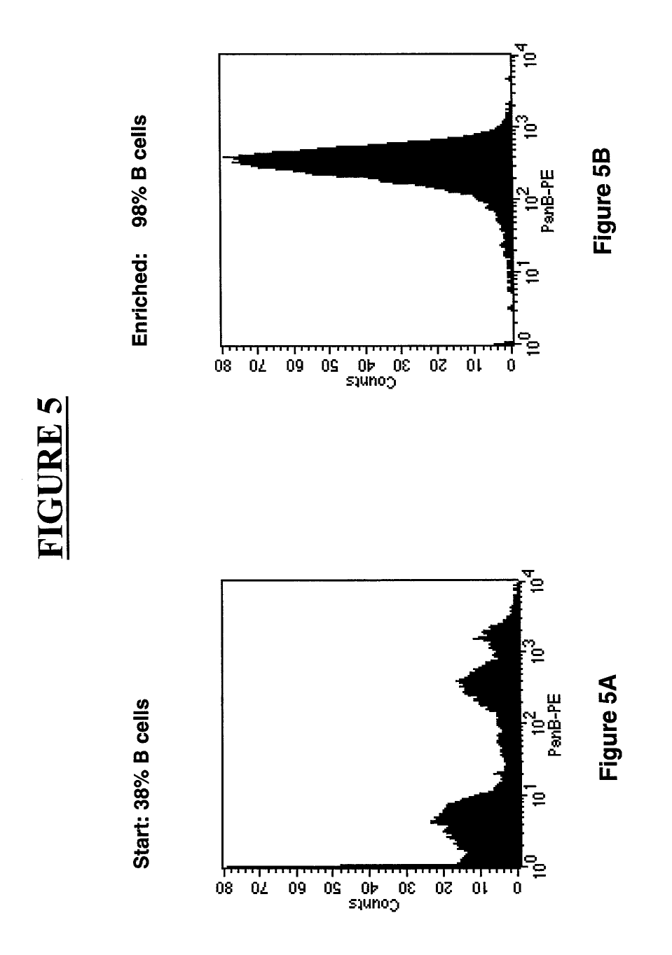

This example demonstrates the enrichment of B cells from rat splenocytes using the method described in Example 1. A B cell enrichment cocktail containing antibodies against OX43, CD161, SIRP, and CD5 or CD4 plus CD8 or CD28 or αβTCR or γδTCR was prepared. The results shown in Table 4 and FIG. 5 demonstrate that the method of the invention results in 98% purity of B cells when using the cocktail containing the anti-CD5 antibody. Substituting the anti-CD5 antibody with antibodies to CD4 and CD8 did not significantly change the purity of B cells.

### Example 7

Isolation of NK Cells from Rat Spleen

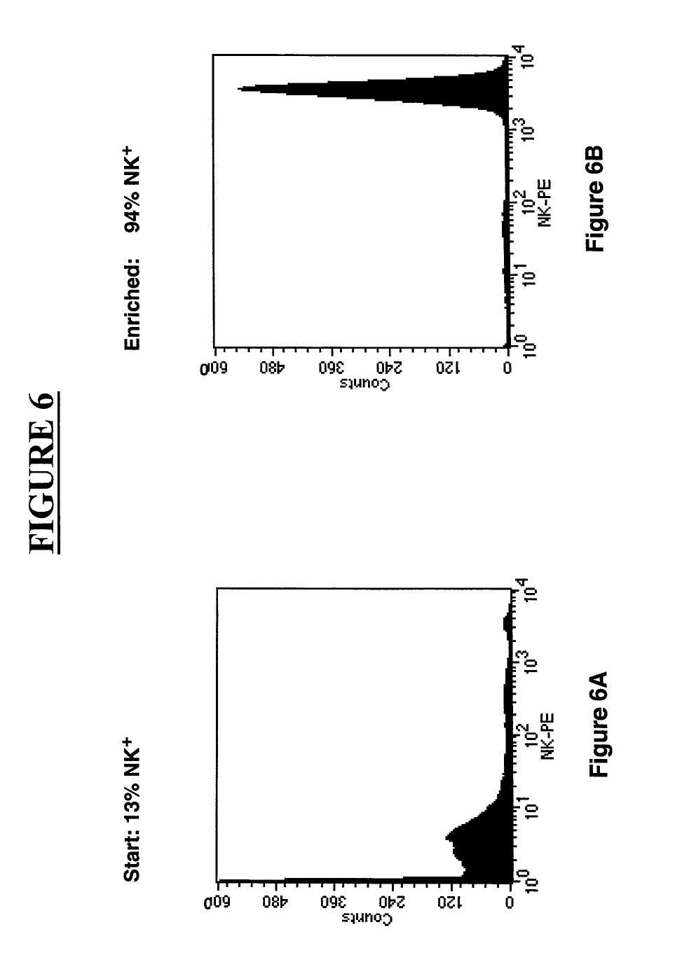

This example demonstrates the enrichment of NK cells from rat splenocytes using the method described in Example 1. An NK cell enrichment cocktail containing antibodies against OX43, CD45RA, SIRP, and CD5 or CD4 plus CD8 or CD28 or αβTCR. The results shown in Table 4 and FIG. 6 demonstrate that the method of the invention results in 94% purity of NK cells when using the cocktail containing the anti-CD5 antibody.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Monoclonal Antibodies to Rat Hematopoietic Cell Surface Markers

| Marker | Clone | Target Cell | Source |
|---|---|---|---|
| OX43 | MRC OX-43 | Red Blood | Serotec Ltd., Oxford, UK |
| antigen | | Cells | |
| CD45RA | MRC OX-33 | B Cells | Serotec Ltd., Oxford, UK |
| SIRP | ED-9 | Myeloid Cells | Serotec Ltd., Oxford, UK |
| CD161 | 10-78 | NK Cells | Serotec Ltd., Oxford, UK |
| CD8 | MRC OX-8 | CD8+ T Cells | Serotec Ltd., Oxford, UK |
| CD4 | W3/25 | CD4+ T Cells | Serotec Ltd., Oxford, UK |
| CD5 | MRC OX-19 | T Cells | Serotec Ltd., Oxford, UK |
| TCRαβ | R73 | T Cells | BD/Pharmingen, |
| | | | La Jolla, CA, USA |
| CD45RC | OX22 | Memory Acti- | BD/Pharmingen, |
| | | vated T Cells | La Jolla, CA, USA |
| CD11b | OX-42 | Myeloid cells | Serotec Ltd., Oxford, UK |
| 1gM | MARM-4 | B cells | Serotec Ltd., Oxford, UK |
| B cells | RNL9D3 | B cells | Serotec Ltd., Oxford, UK |
| MOM/3F12/ | MOM/3F12/F2 | granulocytes | Serotec Ltd., Oxford, UK |
| F2 | | | |
| OX62 | MRC OX-62 | Dendritic Cells | Serotec Ltd., Oxford, UK |
| antigen | | | |
| HIS36 | HIS36 | Macrophages | BD/Pharmingen, |
| | | | La Jolla, CA, USA |
| HIS48 | HIS48 | granulocytes | Serotec Ltd., Oxford, UK |
| CD2 | MRC OX54 | T and NK cells | Serotec Ltd., Oxford, UK |
| CD3 | IF4 | T cells | Serotec Ltd., Oxford, UK |
| ED1 antigen | ED1 | Monocytes and | Serotec Ltd., Oxford, UK |
| | | Macrophages | |
| OX52 | OX52 | T Cells | Serotec Ltd., Oxford, UK |
| CD28 | JJ319 | T Cells | Serotec Ltd., Oxford, UK |

TABLE 2
Lineage Depletion Cocktails to Enrich for Rat Cells

T Cell Enrichment
Anti-
SIRP and/or CD11b
CD45RA, IgM and/or RNL9D3
CD161
and optionally MOM/3F12/F2, OX62, HIS36, HIS48, OX43
CD4 T cell enrichment
Anti-
CD8
SIRP and/or CD11b
CD45RA, IgM and/or RNL9D3
CD161
and optionally MOM/3F12/F2, OX62, HIS36, HIS48, OX43
CD8 T cell enrichment
Anti-
CD4
SIRP and/or CD11b
CD45RA, IgM and/or RNL9D3
CD161
and optionally MOM/3F12/F2, OX62, HIS36, HIS48, OX43
B cell enrichment
Anti-
TCRαβ, CD5, CD2, CD3, CD28, OX52 and/or CD4 plus CD8
SIRP and/or CD11b
CD161
and optionally MOM/3F12/F2, OX62, HIS36, HIS48, OX43
NK Enrichment
Anti-
TCRαβ, CD3 and/or CD28
SIRP and/or CD11b
CD45RA, IgM and/or RNL9D3
and optionally MOM/3F12/F2, OX62, HIS36, HIS48, OX43
Monocyte enrichment
Anti-
TCRαβ, CD5, CD2, CD3 and/or OX52
CD161
CD45RA and/or IgM, RNL9D3
MOM/3F12/F2 and/or HIS48
and optionally OX43, HIS36
Dendritic Cell enrichment
Anti-
TCRαβ, CD5, CD2, CD3 and/or OX52
CD161
CD45RA and/or IgM, RNL9D3
ED1
and optionally MOM/3F12/F2 and/or HIS48, OX43
Hematopoietic Progenitor Enrichment
Anti-
TCRαβ, CD5, CD2, CD3, OX52 and/or CD4 plus CD8
CD161
CD11b and/or SIRP
CD45RA and/or RNL9D3
and optionally OX62, OX43, MOM/3F12/F2, HIS36, HIS48
Naive CD4 T cell enrichment
Anti-
CD8
SIRP and/or CD11b
CD45RA, IgM and/or RNL9D3
CD161
CD45RC
and optionally MOM/3F12/F2, OX62, HIS36, HIS48, OX43
Resting CD4 T cell enrichment
Anti-
CD8
SIRP and/or CD11b
CD45RA, IgM and/or RNL9D3
CD161
CD25 and/or CD45RC
and optionally MOM/3F12/F2, OX62, HIS36, HIS48, OX43
Resting CD8 T cell enrichment
Anti-
CD4
SIRP and or CD11b
CD45RA, IgM and/or RNL9D3
CD25
CD161
and optionally MOM/3F12/F2, OX62, HIS36, HIS48, OX43
γδ T cell Enrichment
Anti-
TCRαβ
SIRP and/or CD11b
CD45RA, IgM and/or RNL9D3
CD161
and optionally MOM/3F12/F2, OX62, HIS36, HIS48, OX43
αβ T cell Enrichment
Anti-
TCRγδ
SIRP and/or CD11b
CD45RA, IgM and/or RNL9D3
CD161
and optionally MOM/3F12/F2, OX62, HIS36, HIS48, OX43

TABLE 3
Isolation of Rat T Cells

| | % in Star | % in Enriched | |
|---|---|---|---|
| | Suspension | Fraction | % Recovery |
| Cell type | mean ± 1 SD | mean ± 1 SD | mean ± 1 SD |
| CD4+ T cell | 25 ± 7 | 96 ± 2 | 29 ± 6 |
| CD8+ T Cell | 17 ± 5 | 95 ± 2 | 30 ± 10 |
| Cell | 41 ± 6 | 99 ± 1 | 35 ± 6 |

n = 8

TABLE 4
Isolation of B cells and NK cells

| | | % | |
|---|---|---|---|
| | % in Start | in Enriched | |
| Cell type | suspension | fraction | Antibodies in cocktail |
| B cells | 39 | 98 | OX43, CD161, SIRP, CD5 |
| | 38 | 97 | OX43, CD161, SIRP, CD4, CD8 |
| | 38 | 95 | OX43, CD161, SIRP, CD28 |
| | 34 | 40 | OX43, CD161, SIRP, αβTCR |
| | 34 | 5.0 | OX43, CD161, SIRP, γδTCR |
| NK cells | 12 | 94 | OX43, CD45RA, SIRP, CD5 |
| | 12 | 78 | OX43, CD45RA, SIRP, CD4, CD8 |
| | 12 | 69 | OX43, CD45RA, SIRP, CD28 |
| | 13 | 64 | OX43, CD45RA, SIRP, αβTCR |

We claim:

1. An antibody composition for enriching rat T cells comprising antibodies specific for the antigens (a) SIRP and/or CD11b; (b) CD45RA and/or IgM and/or RNL9D3; and (c) CD161.

2. An antibody composition according to claim 1 further including antibodies to MOM/3F12/F2, OX62, HIS36, HIS48 and/or OX43.

3. An antibody composition according to claim 2 comprising antibodies specific for SIRP, CD45RA, CD161 and OX43.

4. An antibody composition according to claim 1 for enriching rat CD4⁺ T cells comprising antibodies specific for the antigens (a) CD8; (b) SIRP and/or CD11b; (c) CD45RA and/or IgM and/or RNL9D3; and (d) CD161.

5. An antibody composition according to claim 4 further including antibodies to MOM/3F12/F2, OX62, HIS36, HIS48 and/or OX43.

6. An antibody composition according to claim 5 comprising antibodies specific for SIRP, CD45RA, CD161, OX43 and CD4.

7. An antibody composition according to claim 1 for enriching rat CD8⁺ T cells comprising antibodies specific for the antigens (a) CD4; (b) SIRP and/or CD11b; (c) CD45RA and/or IgM and/or RNL9D3; and (d) CD161.

8. An antibody composition according to claim 7 further including antibodies to MOM/3F12/F2, OX62, HIS36, HIS48 and/or OX43.

9. An antibody composition according to claim 8 comprising antibodies specific for SIRP, CD45RA, CD161, OX43 and CD8.

10. An antibody composition according to claim 1 for enriching rat naive CD4⁺ T cells comprising antibodies specific for the antigens (a) CD8; (b) SIRP and/or CD11b; (c) CD45RA and/or IgM and/or RNL9D3; (d) CD161; and (e) CD45RC.

11. An antibody composition according to claim 10 further including antibodies to MOM/3F12/F2, OX62, HIS36, HIS48 and/or OX43.

12. An antibody composition according to claim 1 for enriching rat resting CD4⁺ T cells comprising antibodies specific for the antigens (a) CD8; (b) SIRP and/or CD11b; (c) CD45RA and/or IgM and/or RNL9D3; (d) CD161; and (e) CD25 and/or CD45RC.

13. An antibody composition according to claim 12 further including antibodies to MOM/3F12/F2, OX62, HIS36, HIS48 and/or OX43.

14. An antibody composition according to claim 1 for enriching rat resting CD8⁺ T cells comprising antibodies specific for the antigens (a) CD4; (b) SIRP and/or CD11b; (c) CD45RA and/or IgM and/or RNL9D3; (d) CD25; and (e) CD161.

15. An antibody composition according to claim 14 further including antibodies to MOM/3F12/F2, OX62, HIS36, HIS48 and/or OX43.

16. An antibody composition according to claim 1 for enriching γδ T cells comprising antibodies specific for the antigens (a) TCRαβ; (b) SIRP and/or CD11b; (c) CD45RA and/or IgM and/or RNL9D3; and (d) CD161.

17. An antibody composition according to claim 16 further including antibodies to MOM/3F12/F2, OX62, HIS36, HIS48 and/or OX43.

18. An antibody composition according to claim 1 for enriching rat αβ T cells comprising antibodies specific for the antigens (a) TCRγδ; (b) SIRP and/or CD11b; (c) CD45RA and/or IgM and/or RNL9D3; and (d) CD161.

19. An antibody composition according to claim 18 further including antibodies to MOM/3F12/F2, OX62, HIS36, HIS48 and/or OX43.

20. An antibody composition for enriching rat B cells comprising antibodies specific for the antigens (a) (i) TCRαβ and/or (ii) CD5 and/or (iii) CD2 and/or (iv) CD3 and/or (v) CD28 and/or (vi) OX52 and/or (vii) CD4 and CD8; (b) SIRP and/or CD11b; and (c) CD161.

21. An antibody composition according to claim 20 further including antibodies to MOM/3F12/F2, OX62, HIS36, HIS48 and/or OX43.

22. An antibody composition according to claim 21 comprising antibodies specific for the antigens (a) (i) CD5 and/or (ii) CD4 and CD8 and/or (iii) CD28; (b) SIRP; (c) CD161 and (d) OX43.

23. An antibody composition for enriching rat NK cells comprising antibodies specific for the antigens (a) TCRαβ and/or CD3 and/or CD28; (b) SIRP and/or CD11b; and (c) CD45RA and/or IgM and/or RNL9D3.

24. An antibody composition according to claim 23 further including antibodies to MOM/3F12/F2, OX62, HIS36, HIS48 and/or OX43.

25. An antibody composition according to claim 24 comprising antibodies specific for the antigens (a) CD5; (b) SIRP; (c) CD45RA and (d) OX43.

26. An antibody composition for enriching rat monocyte cells comprising antibodies specific for the antigens (a) TCRαβ and/or CD5 and/or CD2 and/or CD3 and/or OX52; (b) CD161; (c) CD45RA and/or IgM and/or RNL9D3; and (d) MOM/3F12/F2 and/or HIS48.

27. An antibody composition according to claim 26 further including antibodies to OX43 and/or HIS36.

28. An antibody composition for enriching rat dendritic cells comprising antibodies specific for the antigens (a) TCRαβ and/or CD5 and/or CD2 and/or CD3 and/or OX52; (b) CD161; (c) CD45RA and/or IgM and/or RNL9D3; and (d) ED1.

29. An antibody composition according to claim 28 further including antibodies to MOM/3F12/F2, HIS48 and/or OX43.

30. An antibody composition for enriching rat hematopoietic progenitor cells comprising antibodies specific for the antigens (a) (i) TCRαβ and/or (ii) CD5 and/or (iii) CD2 and/or (iv) CD3 and/or (v) OX52 and/or (vi) CD4 and CD8; (b) CD161; (c) CD11b and/or SIRP; and (d) CD45RA and/or RNL9D3.

31. An antibody composition according to claim 30 further inlcuding antibodies to MOM/3F12/F2, HIS48, HIS36, OX62 and/or OX43.

32. An antibody composition according to claim 1, wherein the antibodies are monoclonal antibodies.

33. An antibody composition according to claim 32 wherein the antibodies are labelled with a marker or they are directly or indirectly conjugated to a matrix.

34. An antibody composition according to claim 32 wherein the antibodies are labelled with biotin or a fluorochrome.

35. An antibody composition according to claim 33 wherein the matrix is magnetic beads, a panning surface, dense particles for density centrifugation, an adsorption column, or an adsorption membrane.

36. An antibody composition according to claim 32 wherein each of the monoclonal antibodies is incorporated in a tetrameric antibody complex which comprises a first monoclonal antibody of a first animal species from the antibody composition according to claim 32, and a second monoclonal antibody of the first animal species which is capable of binding to at least one antigen on the surface of a matrix, which have been conjugated to form a cyclic tetramer with two monoclonal antibodies of a second animal species directed against the Fc-fragments of the antibodies of the first animal species.

37. A negative selection process for enriching and recovering rat T cells in a sample comprising (1) reacting the sample with an antibody composition containing antibodies capable of binding to the antigens (a) SIRP and/or CD11b; (b) CD45RA and/or IgM and/or RNL9D3; and (c) CD161 under conditions permitting the formation of conjugates between the antibodies and cells in the sample having the antigens (a) SIRP and/or CD11b; (b) CD45RA and/or IgM and/or RNL9D3; and (c) CD161 on their surfaces; (2) removing the conjugates; and (3) recovering a cell preparation which is enriched in T cells.

38. A negative selection process according to claim 37 for enriching and recovering rat CD4⁺ T cells in a sample comprising (1) reacting the sample with an antibody composition containing antibodies capable of binding to the antigens (a) CD8; (b) SIRP and/or CD11b; (c) CD45RA and/or IgM and/or RNL9D3; and (d) CD161 under conditions permitting the formation of conjugates between the antibodies and cells in the sample having the antigens (a) CD8; (b) SIRP and/or CD11b; (c) CD45RA and/or IgM and/or RNL9D3; and (d) CD161 on their surfaces; (2) removing the conjugates; and (3) recovering a cell preparation which is enriched in rat CD4⁺ T cells.

39. A negative selection process according to claim 37 for enriching and recovering rat CD8⁺ T cells in a sample comprising (1) reacting the sample with an antibody composition containing antibodies capable of binding to the antigens (a) CD4; (b) SIRP and/or CD1 b; (c) CD45RA and/or IgM and/or RNL9D3; and (d) CD161 under conditions permitting the formation of conjugates between the antibodies and cells in the sample having the antigens (a) CD4; (b) SIRP and/or CD11b; (c) CD45RA and/or IgM and/or RNL9D3; and (d) CD161 on their surfaces; (2) removing the conjugates; and (3) recovering a cell preparation which is enriched in rat CD8⁺ T cells.

40. A negative selection process according to claim 37 for enriching and recovering rat naive CD4⁺ T cells in a sample comprising (1) reacting the sample with an antibody composition containing antibodies capable of binding to the antigens (a) CD8; (b) SIRP and/or CD11b; (c) CD45RA and/or IgM and/or RNL9D3; (d) CD161; and (e) CD45RC under conditions permitting the formation of conjugates between the antibodies and cells in the sample having the antigens (a) CD8; (b) SIRP and/or CD11b; (c) CD45RA and/or IgM and/or RNL9D3; (d) CD161; and (e) CD45RC on their surfaces; (2) removing the conjugates; and (3) recovering a cell preparation which is enriched in rat naive CD4⁺ T cells.

41. A negative selection process according to claim 37 for enriching and recovering rat resting CD4⁺ T cells in a sample comprising (1) reacting the sample with an antibody composition containing antibodies capable of binding to the antigens (a) CD8; (b) SIRP and/or CD11b; (c) CD45RA and/or IgM and/or RNL9D3; (d) CD161; and (e) CD25 and/or CD45RC under conditions permitting the formation of conjugates between the antibodies and cells in the sample having the antigens (a) CD8; (b) SIRP and/or CD11b; (c) CD45RA and/or IgM and/or RNL9D3; (d) CD161; and (e) CD25 and/or CD45RC on their surfaces; (2) removing the conjugates; and (3)-recovering a cell preparation which is enriched in rat resting CD4⁺ T cells.

42. A negative selection process according to claim 37 for enriching and recovering rat resting CD8⁺ T cells in a sample comprising (1) reacting the sample with an antibody composition containing antibodies capable of binding to the antigens (a) CD4; (b) SIRP and/or CD11b; (c) CD45RA and/or IgM and/or RNL9D3; (d) CD25; and (e) CD161 under conditions permitting the formation of conjugates between the antibodies and cells in the sample having the antigens (a) CD4; (b) SIRP and/or CD11b; (c) CD45RA and/or IgM and/or RNL9D3; (d) CD25; and (e) CD161 on their surfaces; (2) removing the conjugates; and (3) recovering a cell preparation which is enriched in rat resting CD8⁺ T cells.

43. A negative selection process according to claim 37 for enriching and recovering rat γδ T cells in a sample comprising (1) reacting the sample with an antibody composition containing antibodies capable of binding to the antigens (a) TCRαβ; (b) SIRP and/or CD11b; (c) CD45RA and/or IgM and/or RNL9D3; and (d) CD161 under conditions permitting the formation of conjugates between the antibodies and cells in the sample having the antigens (a) TCRαβ; (b) SIRP and/or CD11b; (c) CD45RA and/or IgM and/or RNL9D3; and (d) CD161 on their surfaces; (2) removing the conjugates; and (3) recovering a cell preparation which is enriched in rat γδ T cells.

44. A negative selection process according to claim 37 for enriching and recovering rat αβ T cells in a sample comprising (1) reacting the sample with an antibody composition containing antibodies capable of binding to the antigens (a) TCRγδ; (b) SIRP and/or CD11b; (c) CD45RA and/or IgM and/or RNL9D3; and (d) CD161 under conditions permitting the formation of conjugates between the antibodies and cells in the sample having the antigens (a) TCRγδ; (b) SIRP and/or CD11b; (c) CD45RA and/or IgM and/or RNL9D3; and (d) CD161 on their surfaces; (2) removing the conjugates; and (3) recovering a cell preparation which is enriched in rat αβ T cells.

45. A negative selection process for enriching and recovering rat B cells in a sample comprising (1) reacting the sample with an antibody composition containing antibodies capable of binding to the antigens (a) (i) TCRαβ and/or (ii) CD5 and/or (iii) CD2 and/or (iv) CD3 and/or (v) CD28 and/or (vi) OX52 and/or (vii) CD4 and CD8; (b) SIRP and/or CD11b; and (c) CD161 under conditions permitting the formation of conjugates between the antibodies and cells in the sample having the antigens (a) (i) TCRαβ and/or (ii) CD5 and/or (iii) CD2 and/or (iv) CD3 and/or (v) CD28 and/or (vi) OX52 and/or (vii) CD4 and CD8; (b) SIRP and/or CD11b; and (c) CD161 on their surfaces; (2) removing the conjugates; and (3) recovering a cell preparation which is enriched in rat B cells.

46. A negative selection process for enriching and recovering rat NK cells in a sample comprising (1) reacting the sample with an antibody composition containing antibodies capable of binding to the antigens (a) TCRαβ and/or CD3 and/or CD28; (b) SIRP and/or CD11b; and (c) CD45RA and/or IgM and/or RNL9D3 under conditions permitting the formation of conjugates between the antibodies and cells in the sample having the antigens (a) TCRαβ and/or CD3 and/or CD28; (b) SIRP and/or CD11b; and (c) CD45RA and/or IgM and/or RNL9D3 on their surfaces; (2) removing the conjugates; and (3) recovering a cell preparation which is enriched in rat NK cells.

47. A negative selection process for enriching and recovering rat monocyte cells in a sample comprising (1) reacting the sample with an antibody composition containing antibodies capable of binding to the antigens (a) TCRαβ and/or CD5 and/or CD2 and/or CD3 and/or OX52; (b) CD161; (c) CD45RA and/or IgM and/or RNL9D3; and (d) MOM/3F12/F2 and/or HIS48 under conditions permitting the formation of conjugates between the antibodies and cells in the sample having the antigens (a) TCRαβ and/or CD5 and/or CD2 and/or CD3 and/or OX52; (b) CD161; (c) CD45RA and/or IgM and/or RNL9D3; and (d) MOM/3F12/F2 and/or HIS48 on their surfaces; (2) removing the conjugates; and (3) recovering a cell preparation which is enriched in rat monocyte cells.

48. A negative selection process for enriching and recovering rat dendritic cells in a sample comprising (1) reacting the sample with an antibody composition containing antibodies capable of binding to the antigens (a) TCRαβ and/or CD5 and/or CD2 and/or CD3 and/or OX52; (b) CD161; (c) CD45RA and/or IgM and/or RNL9D3; and (d) ED1 under conditions permitting the formation of conjugates between the antibodies and cells in the sample having the antigens (a) TCRαβ and/or CD5 and/or CD2 and/or CD3 and/or OX52; (b) CD161; (c) CD45RA and/or IgM and/or RNL9D3; and (d) ED1 on their surfaces; (2) removing the conjugates; and (3) recovering a cell preparation which is enriched in rat dendritic cells.

49. A negative selection process for enriching and recovering rat hematopoietic progenitor cells in a sample comprising (1) reacting the sample with an antibody composition containing antibodies capable of binding to the antigens (a) (i) TCRαβ and/or (ii) CD5 and/or (iii) CD2 and/or (iv) CD3 and/or (v) OX52 and/or (iv) CD4 and CD8; (b) CD161; (c) CD11b and/or SIRP; and (d) CD45RA and/or RNL9D3 under conditions permitting the formation of conjugates between the antibodies and cells in the sample having the antigens (a) (i) TCRαβ and/or (ii) CD5 and/or (iii) CD2 and/or (iv) CD3 and/or (v) OX52 and/or (vi) CD4 and CD8; (b) CD161; (c) CD11b and/or SIRP; and (d) CD45RA and/or RNL9D3 on their surfaces; (2) removing the conjugates; and (3) recovering a cell preparation which is enriched in rat hematopoietic progenitor cells.

50. A process according to claim 37, wherein the antibodies in the antibody composition are monoclonal antibodies.

51. A process according to claim 50, wherein the antibodies in the antibody composition are labelled with a marker or they are conjugated to a matrix.

52. A process according to claim 50, wherein the antibodies in the antibody composition are labelled with biotin or a fluorochrome.

53. A process according to claim 50, wherein the matrix is magnetic beads, a panning surface, dense particles for density centrifugation, an adsorption column, or an adsorption membrane.

54. A process according to claim 50, wherein each of the monoclonal antibodies in the antibody composition is incorporated in a tetrameric antibody complex which comprises a first monoclonal antibody of a first animal species from the antibody composition, and a second monoclonal antibody of the first animal species which is capable of binding to at least one antigen on the surface of a matrix, which have been conjugated to form a cyclic tetramer with two monoclonal antibodies of a second animal species directed against the Fc-fragments of the antibodies of the first animal species.

55. A kit useful in preparing a cell preparation enriched in rat T cells comprising antibodies specific for the antigens (a) SIRP and/or CD11b; (b) CD45RA and/or IgM and/or RNL9D3; and (c) CD161, and instructions for preparing a cell preparation enriched in rat T cells.

56. A kit useful in preparing a cell preparation enriched in CD4⁺ T cells comprising antibodies specific for the antigens (a) CD8; (b) SIRP and/or CD11b; (c) CD45RA and/or IgM and/or RNL9D3; and (d) CD161, and instructions for preparing a cell preparation enriched in CD4⁺ T cells.

57. A kit useful in preparing a cell preparation enriched in CD8⁺ T cells comprising antibodies specific for the antigens (a) CD4; (b) SIRP and/or CD11b; (c) CD45RA and/or IgM and/or RNL9D3; and (d) CD161, and instructions for preparing a cell preparation enriched in CD8⁺ T cells.

58. A kit useful in preparing a cell preparation enriched in naive CD4⁺ T cells comprising antibodies specific for the antigens (a) CD8; (b) SIRP and/or CD11b; (c) CD45RA and/or IgM and/or RNL9D3; (d) CD161; and (e) CD45RC, and instructions for preparing a cell preparation enriched in naive CD4⁺ T cells.

59. A kit useful in preparing a cell preparation enriched in resting CD4⁺ T cells comprising antibodies specific for the antigens (a) CD8; (b) SIRP and/or CD11b; (c) CD45RA and/or IgM and/or RNL9D3; (d) CD161; and (e) CD25 and/or CD45RC, and instructions for preparing a cell preparation enriched in resting CD4⁺ T cells.

60. A kit useful in preparing a cell preparation enriched in resting CD8⁺ T cells comprising antibodies specific for the antigens (a) CD4; (b) SIRP and/or CD11b; (c) CD45RA and/or IgM and/or RNL9D3; (d) CD25; and (e) CD161, and instructions for preparing a cell preparation enriched in resting CD8⁺ T cells.

61. A kit useful in preparing a cell preparation enriched in γδ T cells comprising antibodies specific for the antigens (a) TCRαβ; (b) SIRP and/or CD11b; (c) CD45RA and/or IgM and/or RNL9D3; and (d) CD161, and instructions for preparing a cell preparation enriched in γδ T cells.

62. A kit useful in preparing a cell preparation enriched in αβ T cells comprising antibodies specific for the antigens (a) TCRγδ; (b) SIRP and/or CD11b; (c) CD45RA and/or IgM and/or RNL9D3; and (d) CD161, and instructions for preparing a cell preparation enriched in γδ T cells.

63. A kit useful in preparing a cell preparation enriched in rat B cells comprising antibodies specific for the antigens (a) (i) TCRαβ and/or (ii) CD5 and/or (iii) CD2 and/or (iv) CD3 and/or (v) CD28 and/or (vi) OX52 and/or (vii) CD4 and CD8; (b) SIRP and/or CD11b; and (c) CD161, and instructions for preparing a cell preparation enriched in rat B cells.

64. A kit useful in preparing a cell preparation enriched in rat NK cells comprising antibodies specific for the antigens (a) TCRαβ and/or CD3 and/or CD28; (b) SIRP and/or CD11b; and (c) CD45RA and/or IgM and/or RNL9D3, and instructions for preparing a cell preparation enriched in rat NK cells.

65. A kit useful in preparing a cell preparation enriched in rat monocyte cells comprising antibodies specific for the antigens (a) TCRαβ and/or CD5 and/or CD2 and/or CD3 and/or OX52; (b) CD161; (c) CD45RA and/or IgM and/or RNL9D3; and (d) MOM/3F12/F2 and/or HIS48, and instructions for preparing a cell preparation enriched in rat monocyte cells.

66. A kit useful in preparing a cell preparation enriched in rat dendritic cells comprising antibodies specific for the antigens (a) TCRαβ and/or CD5 and/or CD2 and/or CD3 and or OX52; (b) CD161; (c) CD45RA and/or IgM and/or RNL9D3; and (d) ED1, and instructions for preparing a cell preparation enriched in rat dendritic cells.

67. A kit useful in preparing a cell preparation enriched in rat hematopoietic progenitor cells comprising antibodies specific for the antigens (a) (i) TCRαβ and/or (ii) CD5 and/or (iii) CD2 and/or (iv) CD3 and/or (v) OX52 and/or (vi) CD4 and CD8; (b) CD161; (c) CD11b and/or SIRP; and (d) CD45RA and/or RNL9D3, and instructions for preparing a cell preparation enriched in rat hematopoietic progenitor cells.
